(19) United States
(12) Patent Application Publication
(10) Pub. No.: US 2006/0040251 A1
(43) Pub. Date: Feb. 23, 2006

(54) Portable medical measurement device and method

(76) Inventors: Shiow-Chen Chen Wang, Chu-Nan (TW); Tsai-Yi Chen, Chu-Nan (TW); Tsong-Tseh Tsay, Chu-Nan (TW); Jih-Hsing Yeh, Chu-Nan (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 11/134,408

(22) Filed: May 23, 2005

(30) Foreign Application Priority Data
Aug. 17, 2004 (TW) 93124739

(51) Int. Cl. C12Q 1/00; A61B 5/00; A61B 5/02

(52) U.S. Cl. 435/4; 600/301; 600/485

(57) ABSTRACT

A portable medical measurement device and method are used for biomedical measurements. The medical measurement device comprises a medical measurement unit for analyzing the reaction of a measured target, a signal receiving unit for receiving a measurement signal, a lookup table for storing reference signals and corresponding parameters, and a microprocessor. When making medical measurement, a measured value of the target is analyzed. Next, a measurement signal is received. The lookup table is then used to find out the corresponding parameter of the measurement signal. Finally, the reaction result of the measured value is calculated out by using the corresponding parameter.

### BACKGROUND OF THE INVENTION

1. Field of the Invention

The present invention relates to a portable medical measurement device and method and, more particularly, to a medical measurement device and method, which make use of an inputted signal matched with a lookup table to find out corresponding parameters.

2. Description of Related Art

Along with progress of the bio-chemical industry and development of the electronic industry, the functions of portable medical measurement devices become more and more diversified. Through convenient operation of portable medical measurement devices, medical measurement data can be more easily and quickly obtained in families and hospitals.

The bio-chemical industry and bio-chemical materials are still at their infancy. Most of portable medical products make use of electrochemical reactions for measurement. Electrochemical signals measured by electrochemical electrode strips may differ for the same concentration because there are differences in material properties of the electrochemical electrode strips. The differences may result from the manufacturing process or different standards to cause errors in the preparation of drugs or the enzymes used.

In order to make up for the influence to each batch of electrochemical electrode strips caused by the above errors, an operation mechanism for correcting parameters should be added so that electrochemical electrode strips of the same kind can be used in the same portable medical products for getting the same operation results. This operation mechanism for correcting parameters is illustrated in the disclosure of R.O.C. Pat. No. 559,660 “a portable multi-function electrochemical bio-detection device”. As shown in FIG. 1, an external message memory 72 memories parameter messages required for a concentration sensor 74 to measure the concentrations of different targets to be measured. The external message memory 72 can thus be used to measure the concentrations of targets of different kinds. The external message memory 72 is included in an external message card (not shown), which is inserted in a slot of a portable multi-function electrochemical bio-detection device. Data in the external message memory 72 can be read by a microprocessor 70 via a bus 73.

In practical operation, the microprocessor 70 will first transfer all parameter messages in the external message memory 72 to an electrically erasable programmable read-only memory (EEPROM) 71. When electrochemical reaction happens, an electrochemical reaction signal outputted by the concentration sensor 74 and a parameter message provided by the EEPROM 71 will be combined together with a temperature offset built by a temperature sensor 75, thereby calculating out the concentration of the target. Finally, the calculation result is outputted to a display device 76.

For the above conventional portable multi-function electrochemical bio-detection device, related parameter messages are obtained by means of an external card. The parameter messages are used as correction parameters for adjusting the concentration sensor 74. Because the required parameter messages are stored in the external message card, it is necessary to provide an external memory for storage of the parameter messages. Because an EEPROM is used as the external memory and its contacts may be subject to interference of outside noises, data may be rewritten to cause abnormal test results and even result in consequences difficult to make up for.

### SUMMARY OF THE INVENTION

The primary object of the present invention is to provide a portable medical measurement device and method, which find out correction parameters required for each batch of electrochemical electrode strips by looking up a table to enhance the reliability of the provided parameters.

To achieve the above object, the present invention provides a portable medical measurement device, which at least comprises a medical measurement unit for analyzing the reaction of a measured target to output a measured value, a signal receiving unit for receiving a measurement signal, a lookup table for storing at least a set of data including a reference signal and a corresponding parameter, and a microprocessor for comparing the measurement signal with the reference signals in the lookup table to find out a corresponding parameter of the measurement signal and perform calculation to the corresponding parameter of the measurement signal and the measured value for obtaining a real reaction result of the target.

In order to achieve the above object, the present invention also provides a portable medical measurement method used in a portable medical measurement device. The portable medical measurement device comprises a medical measurement unit and a signal receiving unit. The measurement method comprises the steps of: using the medical measurement unit to analyze the reaction of a measured target for outputting a measured value; using the signal receiving unit to receive a measurement signal; comparing the measurement signal with a reference signal in a lookup table to find out a corresponding parameter of the measurement signal; and calculating the reaction result of the target by using the corresponding parameter of the measurement signal and the measured value. The lookup table is used to store at least a set of data including the reference signal and the corresponding parameter.

The various objects and advantages of the present invention will be more readily understood from the following detailed description when read in conjunction with the appended drawing, in which:

### BRIEF DESCRIPTION OF THE DRAWINGS

FIG. 1 is a circuit block diagram of a portable multi-function electrochemical bio-detection device in the prior art;

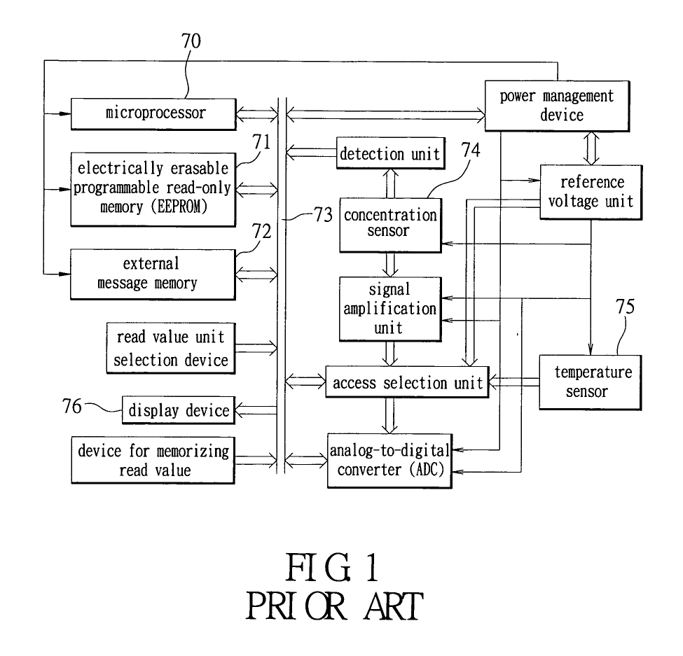

FIG. 2 is a perspective view of a portable medical measurement device of the present invention;

FIG. 3 is a block diagram according to a preferred embodiment of the present invention;

FIGS. 4A to 4C are diagrams of signal cards used in the present invention;

FIG. 5 is a diagram of a lookup table used in the present invention; and

FIG. 6 is a flowchart showing how a portable medical measurement device of the present invention makes use of the lookup table.

### DETAILED DESCRIPTION OF THE PREFERRED EMBODIMENTS

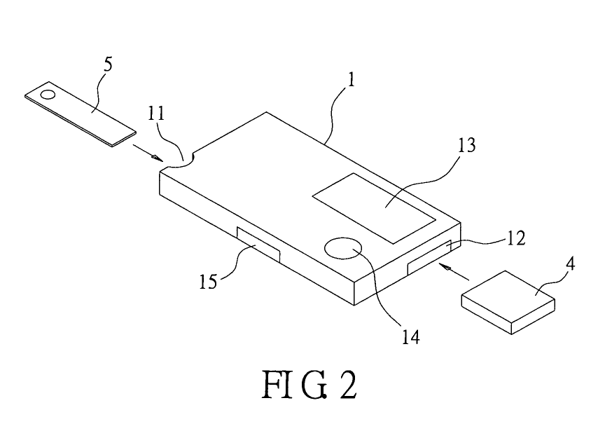

As shown in FIG. 2, an electrochemical electrode strip 5 can be inserted into a strip slot 11 of a medical measurement device 1 to measure the electrochemical reaction of a measured object on the electrochemical electrode strip 5. A signal card 4 can also be inserted into a card reader 12 for input of external signals. Matched with the operation of a key 14, the measured result can be known from a display panel 13. The measured result can also be sent out to a computer, a personal digital assistant (PDA), or a printer via a transmission port 15.

For the medical measurement device 1 of the present invention, the signal card 4 can generate specific measurement signals like square wave, sine wave, triangular wave, periodic signal, non-periodic signal or DC level signal. A lookup table is built in the medical measurement device 1. This lookup table stores at least a set of data including a reference signal and a corresponding parameter in advance. The medical measurement device 1 finds out a corresponding parameter from the lookup table according to the received measurement signal, and then further makes use of the corresponding parameter matched with a measured value of the electrochemical electrode strip 5 to find out the electrochemical reaction of the measured target.

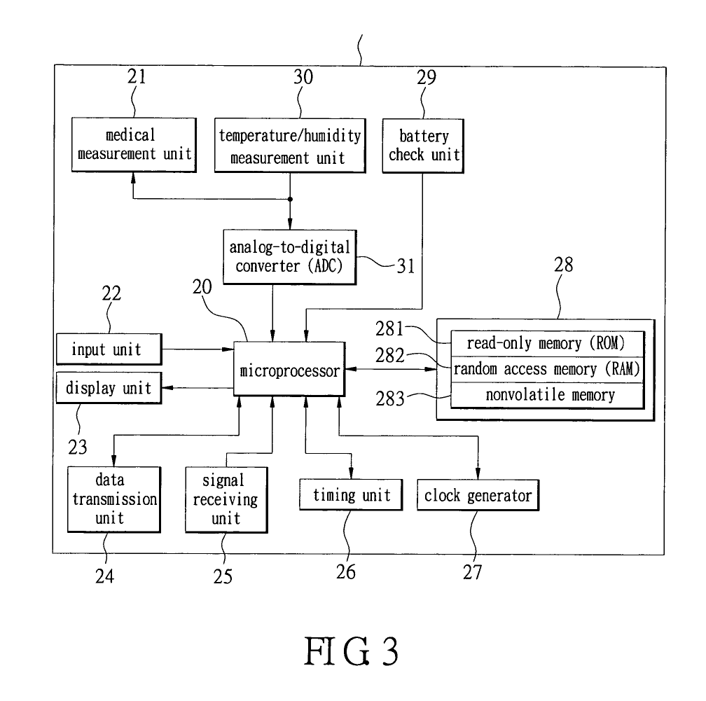

As shown in FIG. 3, the medical measurement device 1 comprises a microprocessor 20, a medical measurement unit 21, an input unit 22, a display unit 23, a data transmission unit 24, a signal receiving unit 25, a timing unit 26, a clock generator 27, a memory unit 28, a battery check unit 29, a temperature/humidity measurement unit 30, and an analog-to-digital converter 31.

The microprocessor 31 is the operation core of the medical measurement device 1, and can provide corresponding operation and processing mechanisms in response to various input commands. The memory unit 28 comprises a read-only memory (ROM) 281, a random access memory (RAM), and a nonvolatile memory 283. The lookup table used in the present invention is stored in the nonvolatile memory 283 or the microprocessor 20. The ROM 281 stores operation codes of the microprocessor 20. The RAM 282 stores temporary data during operation of the microprocessor 20. The nonvolatile memory 283 is preferred to an electrically erasable programmable read-only memory (EEPROM).

In this embodiment, the medical measurement unit 21 is a sensor for measuring blood pressure, blood sugar, cholesterol, HBA₁C, hormone, polymerase chain reaction, medical antibody, or protein. The medical measurement unit 21 performs analysis and measurement to a target to be measured on the electrochemical electrode strip 5 inserted into the strip slot 11, and outputs a measured value. This measured value is an analog value, which should be converted into a digital value by the ADC 31 to be then inputted to the microprocessor 20 for subsequent determination and processing.

The signal receiving unit 25 receives signal data inputted by the signal card 4. In this embodiment, the signal receiving unit 25 is the card reader 12 shown in FIG. 2. The signal receiving unit 25 receives signals in a wired or wireless way. For instance, the signal receiving unit 25 can be a wireless signal receiver or a wired transmission connector. The signal card 4 can be a wireless device of RF, EM wave barcode, or RFID, or can be an electronic device making use of wired transmission like a computer or a PDA.

The data transmission unit 24, like the transmission port 15 shown in FIG. 2, provides a data channel for external transmission. For instance, the processed result of the microprocessor 20 can be sent to a computer, a PDA, or a printer via the data transmission unit 24. The input unit 22, like the key 14 shown in FIG. 2, provides an operation interface for user. The display unit 23, like the display panel 13 shown in FIG. 2, displays messages related to the operation process. The clock generator 27 provides a working clock signal for the microprocessor 20. The timing unit 26 is used for keeping time. The temperature/humidity measurement unit 30 measures the temperature/humidity around the medical measurement device 20. The battery check unit 29 is used to check the electricity of the medical measurement device 20.

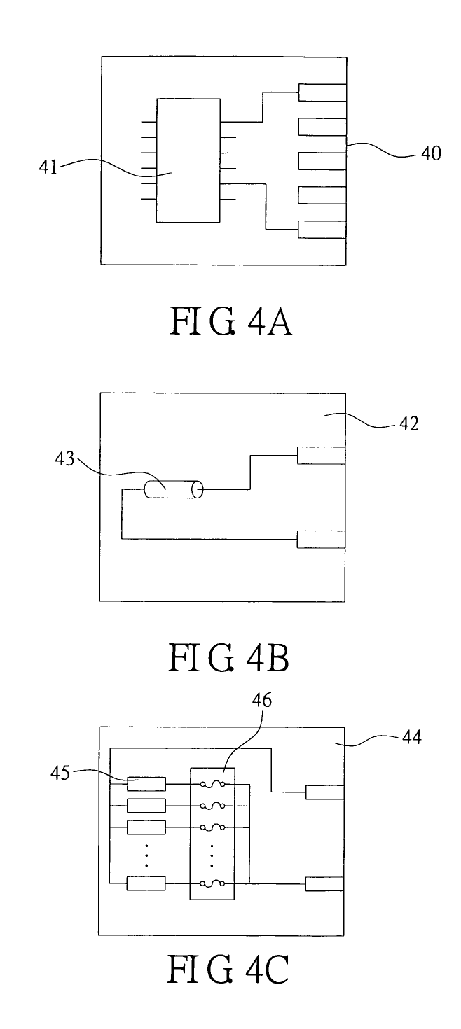

The types of the signal card 4 used in the present invention are shown in FIGS. 4A to 4C. The signal card 4 is primarily realized by providing a component for generating a measurement signal on a printed circuit board (PCB). As shown in FIG. 4A, an IC 41 is disposed on a signal card 40. This IC 41 can produce a square wave, a sine wave, a phase wave, a triangular wave, a periodic signal, or a non-periodic wave. As shown in FIG. 4B, a resistor 43 is disposed on a signal card 42. This resistor 43 is used to produce a constant DC level signal. As shown in FIG. 4C, several resistors 45 of different resistance values are disposed on a signal card 4. Each of the resistors 45 connects to a fuse 46 so that more DC level signals can be provided for selection. Only the fuse corresponding to the wanted DC level signal is reserved while all other fuses are burned out for generate a measurement signal.

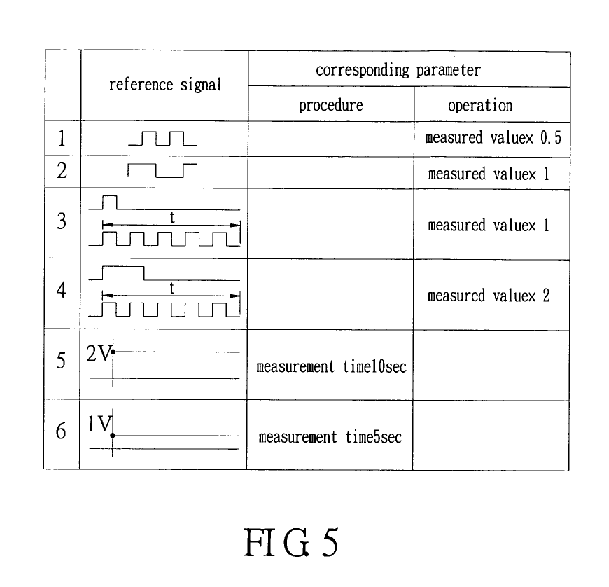

As shown in FIG. 5, the lookup table includes reference signals and corresponding parameter data. Each reference signal has a corresponding parameter. The corresponding parameters can be specific procedures or operations. As for a specific procedure, an original measurement time Of 10 sec can be changed to 9 sec based on different signals. As for an operation, an original multiplication by 1 can be changed to a multiplication by 2 based on different signals. The corresponding parameters can also have other uses. For instance, an original measurement of blood sugar can be changed to a measurement of uric acid according to the corresponding content of the lookup table for different control modes, or a standard product can be changed to an OEM product according to the corresponding content of the lookup table for different judgment conditions.

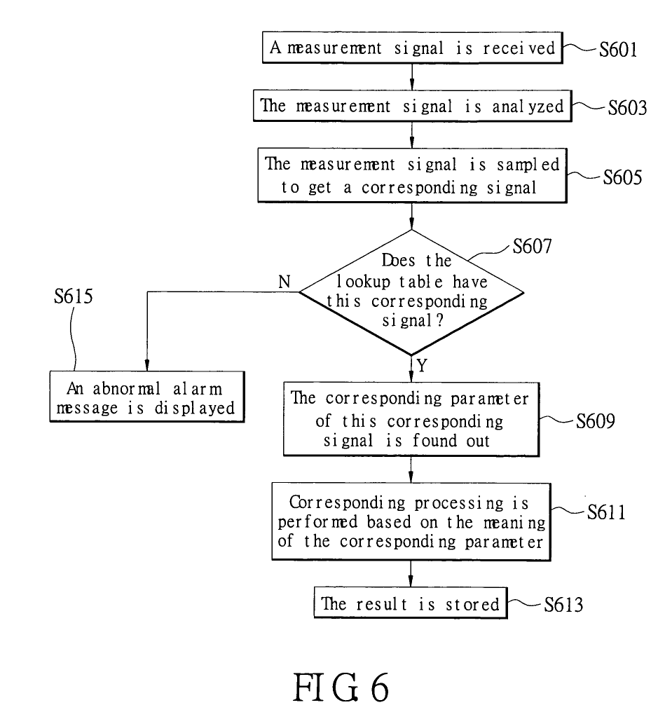

FIG. 6 is a flowchart showing how a portable medical measurement device of the present invention makes use of the lookup table. When the medical measurement device 1 has an electrochemical electrode strip 5 inserted therein and analyzes the reaction of a measured target to output a measured value, an operation mechanism for correcting parameters should be added. The present invention specially provides a table lookup method to find out the corresponding parameter needed to be corrected. This method comprises the following steps.

First, the signal receiving 25 receives a measurement signal (Step S601). This measurement signal is outputted by the signal card 4. Next, the measurement signal is analyzed (Step S603) to discriminate its type (square wave, sine wave, phase wave, triangular wave, periodic signal, non-periodic signal, or DC level signal). The measurement signal is then sampled to get a corresponding signal (Step S605). The sampling way can differ according to the type of the measurement signal. For instance, the sampling can be accomplished by means of digital detection, time sampling detection, periodic time detection, non-periodic time detection, duty-cycle detection, or phase detection. Subsequently, whether the lookup table has this corresponding signal is determined (Step S607). The corresponding signal is compared with reference signals in the lookup table one by one. If the answer is yes, the corresponding parameter of the corresponding signal in the lookup table is found out (Step S609). Corresponding calculation and processing is performed based on the meaning of the corresponding parameter matched with the measured value of the electrochemical electrode strip 5 (Step S611). Finally, the result is stored (Step S613). If the answer in Step S607 is no, an abnormal alarm message is displayed (Step S615).

In Step S609, in addition to being displayed on the display unit 23, the found corresponding parameter can also be manually adjusted through the input unit 23 or the key 14. The adjustable range is predefined in the lookup table, especially when the signal card 4 is as shown in FIG. 4C. This signal card 4 is characterized in that a resistor is selected from several resistors with different resistance values for outputting a specific DC level signal as the measurement signal. Because the user can manually adjust the corresponding parameter found by the table lookup method, the corresponding parameter will have better flexible variation space to conform to diversified inputs of measurement signal.

The medical measurement device 1 of the present invention is used to measure the electrochemical reaction of an electrochemical electrode strip 5. After the medical measurement device 1 detects a periodic, non-periodic, or constant-level measurement signal inputted from the outside, a corresponding parameter of this signal is found out by means of the table lookup method based on the specific meaning of the measurement signal. Therefore, different processing can be performed according to the corresponding parameter matched with the result of the electrochemical reaction.

To sum up, the portable medical measurement device and method of the present invention have the following advantages.

1. The reliability of external signal card can be enhanced.

2. The corresponding parameter is found out by a table lookup method so that corresponding processing can be performed more flexibly based on different characteristics of electrochemical electrode strip.

Furthermore, in addition to being able to measure the electrochemical electrode strips, the medical measurement device can also measure optical electrode strips. Moreover, the insertion position of the signal card 4 is not limited to the card reader 12. The signal card 4 can also be inserted into the strip slot 11. Because the strip slot 11 has the capability of reading electrochemical values, the electric signal outputted by the signal card 4 can be read by the medical measurement unit 21 in the strip slot 11 and then be determined and processed by the microprocessor 20.

Although the present invention has been described with reference to the preferred embodiment thereof, it will be understood that the invention is not limited to the details thereof. Various substitutions and modifications have been suggested in the foregoing description, and other will occur to those of ordinary skill in the art. Therefore, all such substitutions and modifications are intended to be embraced within the scope of the invention as defined in the appended claims.

I claim:

1. A portable medical measurement device at least comprising:
a medical measurement unit for analyzing the reaction of a measured target to output a measured value;
a signal receiving unit for receiving a measurement signal;
a lookup table for storing at least a set of data including a reference signal and a corresponding parameter; and
a microprocessor for comparing said measurement signal with said reference signals in said lookup table to find out a corresponding parameter of said measurement signal and perform calculation to said corresponding parameter of said measurement signal and said measured value for obtaining a real reaction result of said target.

2. The portable medical measurement device as claimed in claim 1, wherein said medical measurement unit is a sensor for measuring blood pressure, blood sugar, cholesterol, HBA₁C, hormone, polymerase chain reaction, medical antibody, or protein.

3. The portable medical measurement device as claimed in claim 1, wherein said measurement signal is a square wave, a sine wave, a phase wave, a triangular wave, a periodic signal, a non-periodic signal, or a DC level signal.

4. The portable medical measurement device as claimed in claim 1, wherein said measurement signal is outputted by a signal card.

5. The portable medical measurement device as claimed in claim 4, wherein said signal card can input a signal to said medical measurement unit or said signal receiving unit so that they can read said signal.

6. The portable medical measurement device as claimed in claim 4, wherein said signal card is realized by providing an IC on a PCB to generate said measurement signal.

7. The portable medical measurement device as claimed in claim 4, wherein said signal card is realized by providing a resistor on a PCB to generate said measurement signal.

8. The portable medical measurement device as claimed in claim 4, wherein said signal card is realized by providing a plurality of resistors on a PCB to generate said measurement signal, each resistor connects to a fuse, and said fuses separately connected with said resistors are burned out except one.

9. The portable medical measurement device as claimed in claim 1, wherein said signal receiving unit is a card reader, a wireless signal receiver, or a connector.

10. The portable medical measurement device as claimed in claim 1 further comprising a temperature/humidity detection unit for measuring the temperature/humidity around said portable medical measurement device.

11. The portable medical measurement device as claimed in claim 1 further comprising a data transmission unit for transmission with the outside.

12. A medical measurement method applied to a portable medical measurement device, said portable medical measurement device comprising a medical measurement unit and a signal receiving unit, said medical measurement method comprising the steps of:
using said medical measurement unit to analyze the reaction of a measured target for outputting a measured value;
using said signal receiving unit to receive a measurement signal; comparing said measurement signal with a reference signal in a lookup table to find out a corresponding parameter of said measurement signal, said lookup table being used to store at least a set of data including said reference signal and said corresponding parameter; and
calculating the reaction result of said target by using said corresponding parameter of said measurement signal and said measured value.

13. The medical measurement method as claimed in claim 12, wherein said measurement signal is a square wave, a sine wave, a phase wave, a triangular wave, a periodic signal, a non-periodic signal, or a DC level signal.

14. The medical measurement method as claimed in claim 12, wherein said measurement signal is outputted by a signal card.

15. The medical measurement method as claimed in claim 14, wherein said signal card makes use of an IC to generate said measurement signal.

16. The medical measurement method as claimed in claim 14, wherein said signal card makes use of a resistor to generate said measurement signal.

17. The medical measurement method as claimed in claim 14, wherein said signal card makes use of a plurality of resistors to generate said measurement signal, each said resistor connects to a fuse, and said fuses separately connected with said resistors are burned out except one.

18. The medical measurement method as claimed in claim 12 further comprising a step of sampling said measurement signal to get a corresponding signal.

19. The medical measurement method as claimed in claim 18, wherein the sampling in said step of sampling said measurement signal to get a corresponding signal is accomplished by means of digital detection, time sampling detection, periodic time detection, non-periodic time detection, duty-cycle detection, or phase detection.

20. The medical measurement method as claimed in claim 12 further comprising a step of storing the result of said step of calculating the reaction result of said target by using said corresponding parameter of said measurement signal and said measured value.
